(19) United States
(12) Patent Application Publication
(10) Pub. No.: US 2024/0309213 A1
(43) Pub. Date: Sep. 19, 2024

(54) Oxide Ceramic With Pigment

(71) Applicant: Ivoclar Vivadent AG, Schaan (LI)

(72) Inventors: Corina Serban, Lindau (DE); Urs Bolle, Koblach (AT); Christin Wildner, Vilters (CH); Frank Rothbrust, Röns (AT); Sebastian Krolikowski, Lachen (CH); Christian Ritzberger, Grabs (CH)

(21) Appl. No.: 18/605,719

(22) Filed: Mar. 14, 2024

(30) Foreign Application Priority Data
Mar. 16, 2023 (EP) 23162381.0

(51) Int. Cl. C09C 1/00; A61C 13/09; A61K 6/818

(52) U.S. Cl. CPC C09C 1/0009; A61C 13/09; A61K 6/818; C01P 2002/34; C01P 2006/63

(57) ABSTRACT

An oxide ceramic containing a pigment, wherein the pigment comprises Al, Cr, and one or more of Y, La, and lanthanides. Also provided is a dental shaped body containing the oxide ceramic with the pigment; a use of the oxide ceramic as a dental material; a use of the dental shaped body for preparing a dental restoration; a process for preparing a dental restoration; and a process for preparing the pigment.

### CROSS-REFERENCE TO RELATED APPLICATIONS

This application claims priority to EP 23162381.0, filed on Mar. 16, 2023, the disclosure of which is incorporated herein by reference in its entirety.

### TECHNICAL FIELD

The invention relates to an oxide ceramic containing a pigment.

### BACKGROUND

Oxide ceramics are highly crystalline ceramic materials that are based on oxide compounds and contain at most a very small amount of glass phase. Typical oxide ceramics are based on ZrO₂, Al₂O₃, TiO₂, MgO and combinations, solid solutions and composites thereof. Because of their advantageous mechanical properties, oxide ceramics and especially zirconia ceramics are widely used for manufacturing dental restorations.

Dental restorations shall not only have advantageous mechanical properties, but also be characterized by an appearance that is as natural as possible. The aim is to imitate the translucency properties of natural tooth material and, in addition, to achieve the best possible color match of the dental restoration with the remaining natural teeth and, if necessary, with the color of the oral mucosa and in particular the gingiva. When imitating the color of natural teeth and natural gingiva, it is in particular necessary to achieve a yellow and a red coloration in the materials.

There are different approaches for coloring oxide ceramics red.

Red colorations of oxide ceramics can be achieved with coloring metal ions and with metal salts or metal oxides formed from such metal ions. Since different metal ions have different coloring properties, different colors and shades can be prepared by mixing different metal ions. However, the toxicity of some metals, such as cadmium and selenium, prevents their use in the manufacture of dental restorations.

It is also possible to color oxide ceramics, such as zirconia, red with erbium and its compounds, especially Er₂O₃. However, very high concentrations of these compounds are required to achieve a color that is clearly perceptible. The shade of these compounds tends to be perceived as pink or rose and is thus clearly different from red regions of the gingiva. In addition, erbium compounds are very expensive, and therefore their use in high concentrations is usually avoided.

Porous oxide ceramics can be colored red by applying highly concentrated solutions based on water-soluble Er³⁺ salts, such as ErCl₃·6 H₂O or Er (NO₃)₃·5 HO, to the surface and infiltrating them into the pores. However, the Er³⁺-based infiltration solutions have a strongly acidic pH, which is often in the range of 1-3. In the case of improper use, there is thus for safety reasons a very high risk that the user could be burned or inhale harmful gases/vapors, such as HCl or HNO₃ vapors, when opening the containers without using a proper fume hood or appropriate room ventilation. Furthermore, such solutions are usually not long-term stable and therefore change their properties over time. Also, uncontrolled evaporation of the solvent can cause changes in concentration over time. Uncontrolled complex formation can also occur, since these solutions usually also contain certain organic compounds. In the worst case, this leads to the precipitation of certain compounds.

The infiltration with staining solution can also result in the staining solution not being homogeneously distributed in the porous zirconia. This can depend on the user as well as on the surface quality of the restoration, such as the degree to which it is free of dust and the moisture content. Inhomogeneities can have a significant impact on the final result.

Infiltration in the gingival area also causes a change in the degree of stabilization and the phase composition of the zirconia after dense sintering. During dense sintering, Er³⁺ ions can be incorporated into the crystal framework in addition to the Y³⁺ ions already incorporated into the zirconia. This then leads in the infiltrated areas to overstabilization and an associated change in the phase composition, which causes a deterioration of mechanical properties, in particular fracture toughness and biaxial strength. In implant-supported restorations a very large chewing load is taken up in particular in the gingival area, which means that a weakening of this area must be avoided.

Furthermore, doping with Er₂O₃ in higher concentrations alters the sintering behavior of zirconia. Er₂O₃ acts as a sintering inhibitor and changes the theoretical density of the stabilized zirconia. The total shrinkage of the infiltrated area is changed, so that the enlargement factor taken into account when milling a blank is no longer correct and the accuracy of fit of the densely sintered restoration is negatively affected. In the case that Er₂O₃ is only used locally for coloring the gingival area, there will be delayed shrinkage locally during sintering, i.e. while the tooth area is already shrinking, the restoration in the gingival area lags behind the shrinkage. This results in stresses during the sintering process, which remain in the overall structure and weaken it permanently. Sudden fractures frequently occur at the working site or during insertion of the restoration.

### SUMMARY

The aim of the invention is therefore to provide oxide ceramics with different red colors. The red colors should, for example, be able to imitate the red colors of the gingiva or natural teeth. In addition, the red colors should exhibit a high temperature stability so that the oxide ceramics can be exposed, for example, to the high temperatures typically used in preparing dental restorations from oxide ceramics. The oxide ceramics should also allow easy and fast processing into dental restorations with very good mechanical properties and in addition should be inexpensive. A health hazard to the persons entrusted with the preparation and to the patients should also be avoided.

This aim of the invention is solved by the oxide ceramic described herein and the dental shaped body described herein. The invention is also directed to the use of the oxide ceramic described herein, the use of the dental shaped body described herein, as well as the process for preparing a dental restoration described herein.

### BRIEF DESCRIPTION OF THE DRAWINGS

Further advantages, details and features will be apparent from the following description of several embodiments of the invention with reference to the drawings.

FIG. 1 shows a schematic perspective view of a multilayer blank according to the invention.

FIG. 2 shows zirconia ceramic platelets of some examples.

### DETAILED DESCRIPTION

The oxide ceramic according to the invention is characterized in that it contains a pigment, wherein the pigment comprises Al, Cr, and Z, and Z is selected from the group consisting of Y, La, lanthanides, and mixtures thereof.

The oxide ceramic of the invention can be given different shades of red color. For example, the oxide ceramic can be given colors that are perceived as colors of natural teeth or of the gingiva. In particular, the oxide ceramic according to the invention allows imitating the red colors of natural teeth and even natural gingiva when preparing dental restorations.

Surprisingly, it has been found that the pigments in oxide ceramics are stable also at the high temperatures typically used in oxide ceramic processing, such as sintering. Therefore, it is particularly advantageous to use the oxide ceramics of the invention in the preparation of dental shaped bodies.

It has been found that the high temperature stability is also present in the microstructure of the oxide ceramic. Furthermore, it has been found that the oxide ceramic according to the invention has mechanical properties like the corresponding conventional oxide ceramics that do not contain the pigment. This is surprising because the fine distribution of pigments in oxide ceramics often results in undesirable effects on the microstructure. In particular, there may be a deterioration in the mechanical properties of the oxide ceramics or undesirable effects during sintering.

It has also been found that an intense red color can be achieved in the oxide ceramics according to the invention in an inexpensive manner.

The ceramics according to the invention are also particularly advantageous in terms of toxicity risk and biocompatibility, since it poses no health risk to the persons entrusted with processing the oxide ceramics and to patients.

According to the invention, the terms “color” and “colored” relate to the color value of a material. Color values can be characterized by the L*a*b* value using a spectrophotometer (e.g. CM 3700-D from Konica-Minolta) according to the DIN 6174 standard or by a shade guide commonly used in the dental industry. The terms “red colors” and “red shades” relate to colors with a positive a* value in the L*a*b* color space.

It is preferred that the pigment comprises Al and Z in a molar ratio of 0.7:1 to 1:0.7, preferably 0.8:1 to 1:0.8 and particularly preferably 0.9:1 to 1:0.9.

In a preferred embodiment, the pigment comprises Z, Al and Cr in a molar ratio corresponding to the formula ZₓAl_{2-x-y}Cr_{y}O₃, wherein x is 0.8 to 1.2, preferably 0.9 to 1.1, in particular 0.95 to 1.05 and particularly preferably 1.00, and y is 0.001 to 0.5, preferably 0.005 to 0.25 and in particular 0.01 to 0.1.

In a further preferred embodiment, the pigment additionally contains Ca.

In a particularly preferred embodiment, the pigment has a composition corresponding to the formula ZₓAl_{2-x-y}Cr_{y}O₃, wherein x is 0.8 to 1.2, preferably 0.9 to 1.1, in particular 0.95 to 1.05 and particularly preferably 1.00, and y is 0.001 to 0.5, preferably 0.005 to 0.25 and in particular 0.01 to 0.1.

In another preferred embodiment, the pigment has a composition corresponding to the formula ZₓAl_{2-x-y}Cr_{y}O₃, wherein Z is selected from the group consisting of yttrium, La and lanthanides, x has the value 1 and y is 0.001 to 0.5. In this case, the pigment has the formula ZₓAl_{1-y}Cr_{y}O₃, wherein Z is selected from the group consisting of yttrium, La and lanthanides and y is 0.001 to 0.5, preferably 0.005 to 0.25 and particularly preferably 0.01 to 0.1.

The molar ratio of Z, Al and Cr in the pigment as well as the composition of the pigment can be determined, for example, by inductively coupled plasma optical emission spectroscopy (ICP-OES), inductively coupled plasma mass spectrometry (ICP-MS) or atomic absorption spectrometry (AAS).

In a preferred embodiment of the oxide ceramic, the main crystal phase of the pigment has a perovskite crystal structure. In this context, the term “main crystal phase” refers to the crystal phase that has the highest mass fraction of all the crystal phases present in the pigment.

The perovskite crystal structure can also be distorted. The term peroxide crystal structure also includes doped perovskite crystals. The pigment can additionally have other crystal phases, such as a garnet structure or a monoclinic crystal structure. For example, when Z is yttrium, the pigment can have a perovskite crystal structure (yttrium-aluminum perovskite, YAP) as the main crystal phase and yttrium-aluminum garnet (YAG) and/or a monoclinic yttrium-aluminum crystal phase (YAM) as additional crystal phases.

The crystal phases present in the pigment can be determined by X-ray diffraction (XRD) analysis. Quantification of the crystal phases can be carried out, in particular, by the Rietveld method.

It is further preferred that Z is selected from the group consisting of Y, Er, Pr, Gd, Dy, Eu, Nd, Yb, Ho and Tm. It has been found that a particularly intense red color with high a* values can be achieved with these pigments in oxide ceramics.

In a preferred embodiment of the oxide ceramic, the pigment has an average particle size d₅₀, determined according to ISO 13320, of 0.05 to 50 μm, in particular 0.1 to 25 μm and particularly preferably 0.5 to 15 μm. Particle sizes in these ranges have been found to be particularly advantageous for the mechanical properties of the oxide ceramics. In particular, it has been found that the use of small particles can lead to a particularly high strength of the oxide ceramic.

In a preferred embodiment, the pigment has a color that has an a* value of at least 7, in particular at least 10. It is particularly preferred that, in addition, the b* value is 5 to 30, in particular 10 to 30, and the L* value is 40 to 65, in particular 45 to 60.

In a further preferred embodiment, the pigment has a color that has an a* value of 7 to 50, a b* value of 10 to 30 and an L* value of 40 to 60. It is particularly preferred that the pigment has a color that has an a* value of 25 to 32, a b* value of 15 to 22, and an L* value of 40 to 60.

It is also preferred that the oxide ceramic has a color with an a* value in the range of 0 to 25, preferably 1 to 20, particularly preferably 2 to 15.

It is further preferred that the oxide ceramic has a color with an a* value in the range from 0 to 13, preferably 1 to 11, particularly preferably 2 to 8. These a* values have been found to be particularly advantageous for imitating tooth color. It is also preferred that the oxide ceramic has a color with an a* value in the range of 5 to 25, preferably 5 to 20, and particularly preferably 5 to 15. It has been found that these a* values are particularly suitable for imitating gingiva color. It is particularly preferred that, in addition to the preferred ranges of a* values, the b* value is 5 to 35, in particular 5 to 25, and the L* value is 65 to 90, in particular 70 to 90.

In a further preferred embodiment, the oxide ceramic contains the pigment in an amount of 0.005 to 10 wt %, in particular 0.01 to 5 wt % and particularly preferably 0.05 to 3 wt %.

It is further preferred that the oxide ceramic contains the pigment in an amount of 0.005 to 5 wt %, in particular 0.01 to 2.5 wt % and particularly preferred 0.05 to 2 wt %. It has been found that these amounts are particularly suitable for imitating tooth color. It is also preferred that the oxide ceramic contains the pigment in an amount of 0.01 to 10 wt %, in particular 0.05 to 5 wt % and particularly preferably 0.1 to 3 wt %. These amounts have proven to be particularly suitable for imitating gingiva color.

The amount of pigment in the oxide ceramic can be determined by combining optical analyses of the microstructures, such as electron microscopy, with chemical analysis techniques, such as energy dispersive X-ray spectroscopy. The proportion of pigment in the oxide ceramic is first determined volumetrically and then converted into a weight proportion, taking into account the densities of the oxide ceramic and the pigment. Crystal phases contained in the pigment can be determined by X-ray diffraction (XRD) analysis.

It is further preferred that the oxide ceramic is a zirconia ceramic, preferably of stabilized tetragonal zirconia polycrystal. Particularly preferably, the zirconia ceramic contains Y₂O₃, La₂O₃, CeO₂, MgO and/or Cao, preferably Y₂O₃ and/or La₂O₃. A zirconia ceramic containing 3 to 5 mol % Y₂O₃ is particularly preferred.

It is also preferred that the oxide ceramic is at least partially sintered. The oxide ceramic is thus preferably either presintered or fully sintered. A presintered oxide ceramic is typically present in an open-pore state. Presintered oxide ceramics allow particularly simple and precise machining. In particular, the lower strength ceramics in the presintered state allows simple, time-saving shaping of blanks with low wear on the milling tool.

Presintered oxide ceramics are subjected to a further sintering step to achieve the desired mechanical properties, in particular high strength and hardness. An oxide ceramic that has been subjected to this further sintering step is referred to as “fully sintered” or also as “densely sintered”. Usually, the density of a densely sintered oxide ceramic, determined according to DIN EN 623-2, is at least 99.2% of the theoretical density of the oxide ceramic.

Dense sintering causes sinter shrinkage of the oxide ceramic. Thus, when using presintered oxide ceramics, it is necessary that the geometry of the oxide ceramic according to the invention is enlarged compared to oxide ceramics based on materials without sinter shrinkage, e.g. plastic materials. Preferably, in the presintered state, the dimensions of the geometry are enlarged by a factor of 1.200 to 1.250, in particular 1.220 to 1.250. In the green state of an oxide ceramic according to the invention, the dimensions of the geometry are preferably increased by a factor of 1.250 to 1.350, in particular about 1.275.

In another preferred embodiment, the oxide ceramic comprises a starting composition of components for the preparation of the pigment described above. In this embodiment, the oxide ceramic is preferably in the green state or partially sintered.

It has been found that an oxide ceramic which is present in the green state or partially sintered state and contains a starting composition of components for the preparation of the pigment regularly exhibits a green color. When such an oxide ceramic is densely sintered, it obtains a red color.

In a preferred embodiment, the oxide ceramic contains 0.005 to 10, preferably 0.01 to 5, and particularly preferably 0.05 to 3 wt % of the starting composition, based on the total weight of the oxide ceramic.

It is preferred that the starting composition contains at least one and preferably all of Al (OH)₃, Cr₂O₃ and Z₂O₃, where Z is selected from Y, La and lanthanides. It is further preferred that the starting composition further comprises one or more flux agents. Suitable flux agents are also referred to as mineralizers and they include fluorides and carbonates of calcium, sodium and barium. In a preferred embodiment, the starting composition contains CaF₂ or a combination of Na₂CO₃ and NaF or a combination of BaCO₃ and NaF, in particular in a molar ratio of 5:1. Particularly preferably, the starting composition contains CaF₂.

It is particularly preferred that the starting composition contains one and preferably all of the following components in the amounts indicated:

| Component | Wt.-% |
|---|---|
| Z₂O₃ | 45 to 65, in particular 50 to 60, |
| Al(OH)₃ | 30 to 45, in particular 35 to 40, |
| Cr₂O₃ | 0.7 to 1.5, in particular 0.9 to 1.3, |
| CaF₂ | 3.0 to 6.0, in particular 4.5 to 5.5, |

wherein Z is selected from Y, La and lanthanides.

The invention further relates to a dental shaped body comprising the oxide ceramic described above.

Preferably, the dental shaped body is a blank or a dental restoration, wherein the dental restoration is in particular selected from crown, partial crown, bridge, abutment, framework, inlay, onlay, veneer, facet, maxillary total prosthesis, mandibular total prosthesis, maxillary partial prosthesis and mandibular partial prosthesis.

The blank according to the invention preferably has a biaxial fracture strength of 10 to 150 MPa, in particular 20 to 120 MPa and more preferably 25 to 80 MPa. The biaxial fracture strength is determined in accordance with ISO 6872 (2008) (piston-on-three-balls test).

It is also preferred that the blank has a fracture toughness of 2 to 10, in particular 2 to 8 and especially preferably 3 to 6. The fracture toughness (K_{Ic}) can be determined by the method described in chapters 5 to 7 of ISO standard 14627, as well as under the test conditions mentioned therein and by using the Niihara equation for Palmquist cracks. The penetration force of the Vickers indenter is 10 kg in this case. The Niihara equation for Palmquist cracks is

$$( \frac{{K}_{Ic} ϕ}{{Ha}^{\frac{1}{2}}} ) {( \frac{H}{E ϕ} )}^{\frac{2}{5}} = 0.035 {( \frac{L}{a} )}^{- 1 / 2}$$

where K_{Ic} is the fracture toughness, ϕ is the constraint factor, H is the hardness, E is the modulus of elasticity, a is the half-diagonal of the Vickers indentation, and furthermore L is described by the equation

$$L = ( \frac{c}{a} - 1 ) * a$$

where a is the half-diagonal of the Vickers indentation and c is the radius of the surface crack. The Niihara equation for Palmquist cracks is also described in K. Niihara et al., Journal of Materials Science Letters, 1 (1982) 13-16.

Furthermore, the blank preferably has a Vickers hardness HV2.5 of 50 to 1000 MPa, in particular 300 to 850 MPa and particularly preferably 300 to 700 MPa. The Vickers hardness is measured according to ISO 14705:2016 at a load of 2.5 kg.

In a preferred embodiment, the dental shaped body is a multilayer blank having a first oxide ceramic layer and a second oxide ceramic layer, wherein the first and second layers differ in color and form an interface.

This multilayer blank according to the invention can comprise two or more oxide ceramic layers, at least one layer of which includes the oxide ceramic according to the invention described above.

The multilayer blank is particularly suitable for the preparation of dental prostheses.

It is preferred that in at least one and in particular in all layers of the multilayer blank the oxide ceramic is a zirconia ceramic.

Preferably, the interface between the first and the second layer in the dental arch of the dental prosthesis to be manufactured is formed in a wave shape with alternating wave troughs and wave crests. This means that in a side view of an arc-shaped, in particular parabolic or semicircular, sectional surface through the blank, the interface between the first and second layers is wave-shaped. In this context, the term “wave shape” is not used restrictively to describe purely sinusoidal undulations only, but generally includes any undulations of alternating elevated and depressed regions.

Furthermore, the vertex lines of the wave crests of the interface between the first layer and the second layer of the multilayer blank preferably extend radially in the mesial-distal direction when viewed in a top view of the interface. That is, in a top view of the interface of the blank, the vertex lines of the wave crests extend from a central region of the blank in a radial direction, i.e., outwardly, in a radiating shape, preferably in the form of straight lines. The three-dimensional wave and radial geometry is preferably based on data from a plurality of real patient cases.

The multilayer blank according to the invention is thus characterized in particular in that the color of teeth and the gingiva as well as the course of the transition from tooth material to gingiva can be imitated particularly well in the final dental prosthesis. The wave form of the first layer can reproduce the gingival margin particularly well. Due to the radially extending wave structure, the gingival margin can always be generated automatically, irrespective of the size of the required dental arch.

In the case of the multilayer blank, the geometry of the interface between the first layer and the second layer of the blank is preferably configured such that the interface, when viewed in a top view of the interface, is fan-shaped in the mesial-distal direction in the region of the anterior teeth of the prosthesis to be manufactured. That is, in a top view of the interface of the blank, the vertex lines of the wave crests extend from a center point of a subsection of the dental arch to be fabricated in a radial direction, i.e., outward, in a fan shape.

Furthermore, it is preferred that the interface between the first layer and the second layer in the region of the molars to be prepared—when viewed in the top view of the interface—has radiating vertex lines of the wave troughs in the oral-buccal direction and, in particular, vertex lines of the wave troughs, and preferably also of the wave crests, that are essentially parallel to one another.

In general, the two above embodiments of a fan-shaped configuration of the vertex lines of the wave crests in the anterior region and a parallel design of the vertex lines of the wave troughs in the posterior region allow both the function and the esthetics of the final dental prosthesis to be improved. In particular, it is possible to provide tooth sets that are suitable for both small and large dental arches. For large dental arches, the dental arch is milled slightly further radially outward, i.e. displaced in the vestibular direction, and for small dental arches, it is milled further inward, i.e. displaced orally. Due to the parallelization of the wave crests and wave troughs of the interface for the region of the molars according to the invention, a comparatively large occlusal surface is also available for small dental arches according to the invention.

Suitable geometries are also known, for example, from EP 3 597 144 A1 and corresponding U.S. Pat. No. 11,684,462 B2, which US patent is hereby incorporated by reference, although the blanks described therein are made of plastic. Compared to these blanks, the multilayer blank according to the invention is characterized in that the use of oxide ceramic layers, and in particular zirconia layers, allows the preparation of high-strength dental restorations which fully meet the requirements with regard to the mechanical properties of, for example, long-span dental restorations, such as prostheses. Furthermore, the use of oxide ceramics compared to plastic allows the formation of thinner blanks, so that the overall height of the blank can be reduced and the machining of the blank in usual CAM milling units can be simplified, e.g. due to fewer undercuts in the design.

Thus, the optical appearance of natural tooth material and natural oral mucosa can be imitated very well with the multilayer blank according to the invention, not only because of the use of the pigment described above. The multilayer blank can also be given the shape of the desired dental prosthesis in a particularly simple manner. The multilayer blank allows efficient monolithic fabrication in the areas of digital fixed and conditionally removable prosthetics, so that a partial or complete denture can be fabricated in one milling process and only a few manual steps. Also, with the blank according to the invention, after shaping, e.g. by CAM milling, no subsequent infiltration with a staining solution in the gingival area is necessary, which in the past has led to frequent cases of failure of the restoration in vivo. This guarantees a stress-free monolithic fabrication and thus a long-term stable restoration.

While the second layer of the blank according to the invention is preferably tooth-colored and retains a tooth color even after heat treatment of the blank, the first layer has the color of the gingiva and retains such a color even after heat treatment.

In a preferred embodiment of the multilayer blank, the oxide ceramic of the first layer contains the pigment in an amount of 0.01 to 10 wt %, in particular 0.05 to 5 wt % and particularly preferably 0.1 to 3 wt %. It is further preferred that the oxide ceramic of the second layer contains the pigment in an amount of 0.005 to 5 wt %, in particular 0.01 to 2.5 wt % and particularly preferred 0.05 to 2 wt %.

Preferably, the oxide ceramic of the first layer has a color, in particular exclusively colors, with an a* value of 5 to 25, in particular 5 to 20 and particularly preferably 5 to 15. It is further preferred that, in addition, the oxide ceramic of the second layer has a color, in particular exclusively colors, with an a* value of 0 to 13, preferably 1 to 11 and in particular 2 to 8.

In a further preferred embodiment of the multilayer blank, the second layer has a continuous, i.e. linear, or discontinuous, i.e. non-linear, color gradient and/or translucency gradient. This also allows the color gradient and translucency gradient of natural teeth, in particular anterior teeth, to be imitated particularly well.

Particularly preferably, the oxide ceramic of the second layer is a zirconia ceramic containing yttrium, and the color gradient and/or translucency gradient, in particular the translucency gradient, is preferably formed by a gradient of the amount of yttrium, i.e. by a gradually changing amount of yttrium. Thus, the blank preferably exhibits a continuous, i.e. linear, or discontinuous, i.e. non-linear, gradient of the amount of yttrium. In particular, the content of yttrium increases from the interface between the first and second layers toward the outer surface of the second layer opposite the interface. This increase in the yttrium content may be without or with steps.

Preferably, the second layer can contain at least one, and more preferably all, of the following components in the indicated amounts:

| Component | Wt. % |
|---|---|
| ZrO₂ + HfO₂ | 82.5-96.5, in particular 84.5-95.0 |
| Y₂O₃ | 3.5-11.0, in particular 5.0-9.0 |
| Pigment | 0.005-5, in particular 0.05-2 |
| Fe₂O₃ | 0-0.15, in particular 0.005-0.12 |
| Cr₂O₃ | 0-0.025, in particular 0.0002-0.01 |
| Mn₂O₃ | 0-0.002, in particular 0.00005-0.0012 |
| Pr₂O₃ | 0-0.02, in particular 0.0001-0.015 |
| Tb₂O₃ | 0-0.02, in particular 0.00005-0.015 |
| Er₂O₃ | 0-1.0, in particular 0.01-0.65 |
| MgO | 0-0.05, in particular 0-0.025 |
| Al₂O₃ | 0-0.25, in particular 0-0.15 |
| La₂O₃ | 0-1.0, in particular 0-0.55 |

The oxide ceramic of the first layer preferably contains zirconia stabilized with yttrium. Preferably, this zirconia ceramic of the first layer has an yttrium content of 0.0 to 4.5 mol %, in particular 0.5 to 4.25 mol %, particularly preferably 0.75 to 2.0 mol %, wherein the yttrium content is defined as the fraction of the amount of Y₂O₃ relative to the sum of the amounts of Y₂O₃, ZrO₂ and HfO₂. Since the first layer of the blank is used to form the gingival area of the dental restoration to be fabricated, formation of a color gradient or material gradient of yttrium in the first layer is not advantageous in many cases. Thus, the yttrium content within first layer is preferably the substantially constant. Alternatively, the first layer may have a gradient intended to represent a fixed and movable gingiva.

Preferably, the oxide ceramic of the first layer contains at least one, and in particular all, of the following components in the amounts indicated:

| Component | Wt.-% |
|---|---|
| ZrO₂ + HfO₂ | 86.0-97.0, |
| | preferably 90.0-94.0 |
| Pigment | 0.01 to 10, |
| | preferably 0.1 to 3 |
| Y₂O₃ | 0.5-8.0, |
| | preferably 1.0-4.0 |
| first coloring oxide | 0.0-0.5, |
| | preferably 0.0-0.25, |
| | particularly preferably 0.0-0.1 |
| second coloring oxide | 0.0-0.1, |
| | preferably 0.0-0.05, |
| | particularly preferably 0.0-0.025, |

wherein the first coloring oxide is selected from the group consisting of Fe₂O₃, Tb₂O₃, Pr₂O₃ and V₂O₅ and can in particular effect an additional yellow color of the oxide ceramic, and wherein the second coloring oxide is selected from the group consisting of Mn₂O₃, Cr₂O₃ and CoO and can in particular effect an additional gray color of the oxide ceramic.

In a further embodiment, it is preferred that the first layer of the multilayer blank comprises zirconia ceramic or a mixture of zirconia ceramic with one or more materials selected from the group consisting of alumina reinforced zirconia, zirconia reinforced alumina, spinels, or mixtures thereof.

Furthermore, it is preferred that in the multilayer blank according to the invention, the first layer and the second layer are joined together by one-piece fabrication. This enables efficient monolithic fabrication, i.e. a patient-specific complete prosthesis can be prepared from one blank in one milling process.

The shape of the multilayer blank according to the invention is preferably at least partially circular. In particular, the blank has the shape of a disk, particularly preferably the shape of a circular disk.

The ratio of the height of the first layer to the height of the second layer is preferably 1:1 to 1:5, in particular 1:2.5 to 1:3.5.

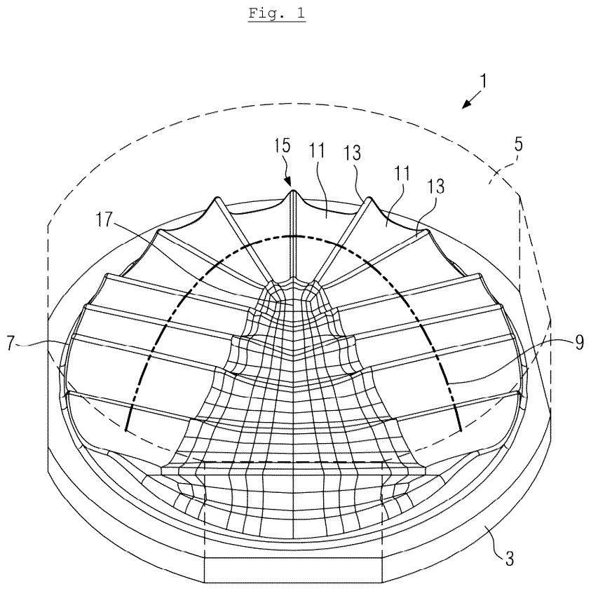

FIG. 1 shows a blank 1 according to the invention for a maxillary prosthesis. The blank 1 is disc-shaped in terms of its basic structure. It has a first layer 3 based in particular on zirconia ceramic and a second layer 5 based in particular on zirconia ceramic. The first layer 3 and the second layer 5 differ in color and form an interface 7. In the representation according to FIG. 1, the upper side of the first layer 3 and the lower side of the second layer 5 represent the interface 7 of the blank. An exemplary schematic course of a dental arch, i.e. the course of the teeth of the prosthesis to be fabricated, is shown in FIG. 1 as line 9. In the course of the dental arch 9, the interface 7 is wave-shaped, with wave troughs 11 alternating with wave crests 13. In the region 15 of the anterior teeth to be fabricated, the wave crests 13 extend in a fan-shaped manner in the oral-vestibular direction. That is, the vertex lines of the wave crests 13 extend from a center point 17 of the dental arch 9 in a radial direction, i.e., outward, toward the vestibular side, in a radial shape.

Furthermore, the invention relates to the use of the oxide ceramic described above as a dental material and in particular for preparing a dental restoration.

In a preferred embodiment, the oxide ceramic containing the starting composition of components for preparing the pigment is subjected to a heat treatment of at least 1200° C., in particular at least 1300° C. and particularly preferably at least 1400° C.

It has surprisingly been shown that in oxide ceramics containing the starting composition of components for preparing the pigments, a red color can be effected by heat treatment at a temperature of at least 1200° C., such as 1450° C. Therefore, the use of an oxide ceramic, which contains the starting composition of components for preparing the pigment, in the preparation of dental restorations has the advantage that the calcination step for preparing the pigment can be omitted, thus saving time and energy.

It is also an object of the invention to use the dental shaped body described above for preparing a dental restoration, which is in particular selected from the group consisting of a crown, bridge, abutment, framework, partial crown, inlay, onlay, veneer, facet, maxillary complete prosthesis, mandibular complete prosthesis, maxillary partial prosthesis and mandibular partial prosthesis. Particularly preferred is the use of the dental shaped body for preparing a maxillary partial prosthesis, mandibular partial prosthesis, maxillary total prosthesis or mandibular total prosthesis.

The use according to the invention can comprise any process steps used for preparing a dental restoration. For example, the use can comprise giving the dental shaped body the shape of the dental restoration.

The invention further relates to a process for preparing a dental restoration, in which the oxide ceramic described above or the dental shaped body described above is processed, and in particular shaped, into a dental restoration.

All of the embodiments of the oxide ceramic, the dental shaped body and the uses described above are also correspondingly suitable or preferred for the process for preparing a dental restoration according to the invention.

The invention is explained in more detail in the following with reference to examples.

### EXAMPLES

### Examples 1 and 2: Oxide ceramics with yttrium pigment

Red colored oxide ceramics according to the invention were prepared.

For preparing the pigment, a starting composition of 37.7 wt % Al (OH) 3, 1.1 wt % Cr₂O₃, 56.2 wt % Y₂O₃ and 5.0 wt % CaF₂ was prepared, mixed and comminuted in an agate mortar. Then, the starting composition was calcined. For this purpose, the starting composition was heated from room temperature to 600° C. within 1 h, then heated from 600° C. to 1300° C. within 2 h, kept at 1300° C. for 1 h, and then cooled freely to room temperature.

Then the obtained pigment was added to a zirconia powder which was stabilized with 5 mol % Y₂O₃ (DKK HSY-0308 5YSZ white). The zirconia powder also contained 3 wt % binder and 0.10 wt % Al₂O₃.

For preparing the oxide ceramic of Example 1, 0.25 wt % pigment were added to 99.75 wt % of the zirconia powder. For preparing the oxide ceramic of Example 2, 0.50 wt % pigment were added to 99.50 wt % of the zirconia powder.

The zirconia powder was mixed with the pigment and then pressed at a pressure of 300 MPa to form platelets (diameter: 16 mm, height: 1.75 mm). The platelets were sintered at 1450° C. for 5 min.

Afterwards, the platelets were treated on both sides with 1000-grit SiC paper.

The L*a*b* color values of the obtained zirconia ceramics were determined according to DIN 6174 using a spectrophotometer (CM 3700-D, Konica-Minolta). The measured values are given in Table 1.

TABLE 1

| Example | L* | a* | b* |
|---|---|---|---|
| 1 | 82.59 | 8.31 | 9.63 |
| 2 | 73.45 | 12.68 | 10.48 |

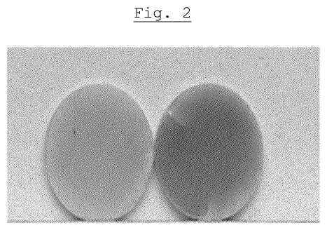

FIG. 2 shows the zirconia ceramic platelets of Examples 1 (left) and 2 (right). The platelets exhibit different shades of red, with the platelet of Example 2 providing a more intense color impression.

It has been found that the platelet of Example 2 exhibits a color that is particularly close to the natural color of the gingiva.

### Examples 3 to 12: Oxide Ceramics with La and Lanthanide Pigments

For Examples 3 to 12, pigments containing lanthanum or lanthanides were initially prepared. For this purpose, different starting compositions were prepared, ground and calcined in accordance with the process of Examples 1 and 2, except that the starting compositions in each case contained 56.2 wt % of the oxides of lanthanum and lanthanides indicated in Table 2 instead of yttria.

Zirconia platelets were then prepared by the process described for Example 2, in which 0.50 wt % of the pigment indicated in Table 2 was added to 99.50 wt % of oxide ceramic. The determination of the L*a*b* color values was also carried out according to the procedure described for Example 2 and the results of the measurements are given in Table 2.

TABLE 2

| Example | Oxide | L* | a* | b* |
|---|---|---|---|---|
| 3 | Er₂O₃ | 70.50 | 14.32 | 8.93 |
| 4 | Gd₂O₃ | 64.13 | 13.82 | 7.35 |
| 5 | Pr₂O₃ | 54.87 | 6.86 | 3.43 |
| 6 | Dy₂O₃ | 66.92 | 12.62 | 6.94 |
| 7 | La₂O₃ | 62.74 | 10.05 | 4.06 |
| 8 | Eu₂O₃ | 62.53 | 11.26 | 4.90 |
| 9 | Nd₂O₃ | 57.92 | 10.12 | 3.60 |
| 10 | Yb₂O₃ | 79.46 | 5.67 | 9.52 |
| 11 | Ho₂O₃ | 67.97 | 13.22 | 9.86 |
| 12 | Tm₂O₃ | 74.29 | 11.60 | 9.05 |

### Examples 13 and 14: Oxide Ceramics with Starting Composition

The zirconia platelets of Examples 13 and 14 were prepared according to the process of Example 1 and examined. However, no pigment was used, but the starting composition of components for preparing the pigment used in Example 1. This means that the starting composition which was mixed and crushed with an agate mortar was added directly to the zirconia powder without calcination.

The process for preparing Example 14 additionally differed in that the platelet was sintered at a temperature of 1500° C.

The starting composition had a green color when it was added to the zirconia powder. After sintering, the zirconia ceramic platelets had a red color. The red color was slightly less intense than in Examples 1 and 2, as can also be seen from the a* values given in Table 3.

TABLE 3

| Example | L* | a* | b* |
|---|---|---|---|
| 13 | 82.90 | 1.39 | 5.11 |
| 14 | 87.48 | 1.90 | 7.41 |

### Example 15: Fracture Toughness of Oxide Ceramics with Yttrium Pigment

Another red colored oxide ceramic according to the invention was prepared using the pigment prepared in Example 1.

A zirconia powder stabilized with 3 mol % Y203 (PU Tosoh Zirconia Zpex) and containing 3.8 wt % binder was used for preparing the oxide ceramic.

0.50 wt % pigment were added to 99.50 wt % zirconia powder and the mixture was homogenized for 30 min in a 3D shaker mixer (Turbula® type, Willy A. Bachofen AG, Switzerland) and then sieved with a 200 μm sieve. The homogenized powder was precompressed into a disc at 720 kN in a 99.6 mm diameter die and then subjected to cold isostatic pressing at 3500 bar. The disc was presintered at 995° C., and then platelets were milled from the disc. The platelets were sintered at 1500° C. for 120 min in a sintering furnace of type Programat S1 1600 (Ivoclar Vivadent AG, Liechtenstein).

Afterwards, the platelets were treated on both sides with 1000-grit SiC paper and the fracture toughness K_{Ic} of the platelets was determined according to the method described in chapters 5 to 7 of ISO 14627, under the test conditions specified therein and with a penetration force of 10 kg, and using the Niihara equation for Palmquist cracks. The measurements showed a maximum fracture toughness of 5.7 MPa·m^{1/2} and an average fracture toughness of 5.27 MPa·m^{1/2}. The fracture toughness of the oxide ceramics colored with pigments was thus not impaired in comparison to corresponding oxide ceramics without pigment.

1. An oxide ceramic comprising
a pigment,
wherein the pigment comprises Al, Cr and Z, and Z is selected from the group consisting of Y, La, lanthanides, and mixtures thereof.

2. The oxide ceramic according to claim 1,
wherein the pigment comprises Al and Z in a molar ratio of 0.7:1 to 1:0.7.

3. The oxide ceramic according to claim 1,
wherein the pigment comprises Z, Al and Cr in a molar ratio corresponding to the formula ZₓAl_{2-x-y}Cr_{y}O₃,
wherein x is 0.8 to 1.2, and y is 0.001 to 0.5.

4. The oxide ceramic according to claim 1,
wherein the main crystal phase of the pigment has a perovskite crystal structure.

5. The oxide ceramic according to claim 1,
wherein Z is selected from the group consisting of Y, Er, Pr, Gd, Dy, Eu, Nd, Yb, Ho and Tm.

6. The oxide ceramic according to claim 1,
which has a color with an a* value in the range from 0 to 25, or in the range from 5 to 25.

7. The oxide ceramic according to claim 1,
which comprises the pigment in an amount of 0.005 to 10 wt %.

8. The oxide ceramic according to claim 1,
which is a zirconia ceramic.

9. The oxide ceramic according to claim 1,
which is at least partially sintered.

10. The oxide ceramic comprising
a starting composition of components for preparing the pigment defined in claim 1.

11. The oxide ceramic according to claim 10,
wherein the starting composition comprises at least one of the components Al (OH) 3, Cr203 and Z203, wherein Z is selected from Y, La and lanthanides.

12. A dental shaped body comprising
the oxide ceramic according to claim 1.

13. The dental shaped body according to claim 12,
wherein the shaped body is a blank or a dental restoration,
wherein the dental restoration is selected from a crown, partial crown, bridge, abutment, framework, inlay, onlay, veneer, facet, maxillary complete prosthesis, mandibular complete prosthesis, maxillary partial prosthesis and mandibular partial prosthesis.

14. The dental shaped body according to claim 13,
which is a multilayer blank having a first oxide ceramic layer and a second oxide ceramic layer,
wherein the first layer and the second layer differ in color and form an interface,
wherein the interface in the course of a dental arch has a wave shape with alternating wave troughs and wave crests, and
wherein the apex lines of the wave crests extend radially in the mesial-distal direction when viewed from a top view of the interface.

15. The dental shaped body according to claim 14,
in which the zirconia ceramic of the first layer has a color with an a* value of 5 to 25.

16. The dental shaped body according to claim 14,
in which the zirconia ceramic of the second layer has a color with an a* value of 0 to 13.

17. A process for preparing a dental restoration,
in which the oxide ceramic according to claim 1 is shaped into a dental restoration.
